(19) United States
(12) Patent Application Publication
(10) Pub. No.: US 2021/0299174 A1
(43) Pub. Date: Sep. 30, 2021

(54) CELL THERAPY

(71) Applicant: M2X2 THERAPEUTICS, INC., Palo Alto, CA (US)

(72) Inventor: Daniel MCHUGH, Palo Alto, CA (US)

(21) Appl. No.: 17/057,206

(22) PCT Filed: May 29, 2019
(86) PCT No.: PCT/US2019/034421

§ 371 Date: Nov. 20, 2020

Related U.S. Application Data
(60) Provisional application No. 62/681,307, filed on Jun. 6, 2018.
(60) Provisional application No. 62/678,043, filed on May 30, 2018.

(51) Int. Cl. A61K 35/17; C12N 15/11; C12N 9/22; C12N 9/02

(52) U.S. Cl. CPC A61K 35/17; C12N 15/11; C12N 2310/20; C12N 9/0071; C12N 9/22

(57) ABSTRACT

Disclosed herein are methods for providing cell therapy for treating or ameliorating a disease in an individual in need thereof, said methods comprising administering to said individual a cellular composition that comprises an engineered T-cell comprising: a first synthetic polynucleotide comprising a sequence encoding a CRISPR nuclease and an epigenetic enzyme or a functional portion thereof that modifies an epigenetic state; and a second synthetic polynucleotide comprising a sequence encoding a guide RNA (gRNA). Further disclosed herein are methods for reducing or preventing T-cell exhaustion in an individual in need thereof, said method comprising administering to said individual a cellular composition that comprises the engineered T-cell.

### CROSS REFERENCE

This application claims the benefit of U.S. Provisional Patent Application No. 62/678,043, filed May 30, 2018, and U.S. Provisional Patent Application No. 62/681,307, filed Jun. 6, 2018, the entire disclosures of which are herein referenced.

### SUMMARY OF THE INVENTION

Disclosed herein, are methods for providing cell therapy for treating or ameliorating a disease in an individual in need thereof, said methods comprising administering to said individual a cellular composition that comprises an engineered T-cell comprising: (a) a first synthetic polynucleotide comprising a sequence encoding (i) a CRISPR nuclease, and (ii) an epigenetic enzyme or a functional portion thereof that modifies an epigenetic state; and (b) a second synthetic polynucleotide comprising a sequence encoding a guide RNA (gRNA). First synthetic polynucleotide may further comprise a sequence encoding (iii) a flexible linker, wherein said linker operably links said sequence encoding (i) and (ii). Epigenetic enzyme may comprise a DNA demethylation enzyme or a DNA hydroxymethylation enzyme (e.g. TET protein such as TET1). Epigenetic enzyme may comprise a DNA methylation enzyme (e.g. DNA methyltransferase (DNMT)). Epigenetic enzyme may comprise a histone acetylation enzyme (e.g. histone acetyltransferase (HAT)). Epigenetic enzyme may comprise a histone deacetylation enzyme (e.g. histone deacetylase (HDAC)). Epigenetic enzyme may comprise a histone methylation enzyme (e.g. histone methyltransferase (HMT)). Epigenetic enzyme may comprise a histone demethylation enzyme (e.g. histone demethylase (HDM)). CRISPR nuclease may be Cas9. CRISPR nuclease may be a deactivated Cas9 (dCas9). First synthetic polynucleotide may further comprise a sequence for a constitutively active promoter. First synthetic polynucleotide may further comprise a sequence for an inducible promoter. gRNA may target a target sequence in said engineered T-cell. Target sequence may comprise a target enhancer sequence, a target regulatory element sequence, a promoter sequence of a target gene, a cis-regulatory sequence of a target gene, or a trans-regulatory sequence of a target gene. Target gene may be a gene that affects T-cell exhaustion. Targeting said target sequence may enhance function of engineered T-cell. Cellular composition may undergo decreased or no T-cell exhaustion, thereby treating or ameliorating disease in said individual. T-cell may be a CAR T-cell. First synthetic polynucleotide and said second synthetic polynucleotide may be encoded on same vector. First synthetic polynucleotide and said second synthetic polynucleotide may be encoded on different vectors. Vector may be a viral vector or a non-viral vector. Disease may be cancer.

Disclosed herein, are methods for providing cell therapy for treating or ameliorating a disease in an individual in need thereof, said method comprising administering to said individual a cellular composition that comprises an engineered T-cell comprising: (a) a first synthetic polynucleotide comprising a sequence encoding (i) a CRISPR nuclease, and (ii) a DNA hydroxymethylation enzyme or a functional portion thereof that modifies DNA methylation state; and (b) a second synthetic polynucleotide comprising a sequence encoding a guide RNA (gRNA). First synthetic polynucleotide may further comprise a sequence encoding (iii) a flexible linker, wherein said linker operably links said sequence encoding (i) and (ii). Enzyme may be a TET protein, such as TET1. CRISPR nuclease may be Cas9. CRISPR nuclease may be a deactivated Cas9 (dCas9). First synthetic polynucleotide may further comprise a sequence for a constitutively active promoter. First synthetic polynucleotide may further comprise a sequence for an inducible promoter. gRNA may target a target sequence in said engineered T-cell. Target sequence may comprise a target enhancer sequence, a target regulatory element sequence, a promoter sequence of a target gene, a cis-regulatory sequence of a target gene, or a trans-regulatory sequence of a target gene. Target gene may be a gene that affects T-cell exhaustion. Targeting said target sequence may enhance function of engineered T-cell. Cellular composition may undergo decreased or no T-cell exhaustion, thereby treating or ameliorating disease in said individual. T-cell may be a CAR T-cell. First synthetic polynucleotide and said second synthetic polynucleotide may be encoded on same vector or on different vectors. Vector may be a viral vector or a non-viral vector. Disease may be cancer.

Disclosed herein, are methods for reducing or preventing T-cell exhaustion in an individual in need thereof, said method comprising administering to said individual a cellular composition that comprises an engineered T-cell comprising: (a) a first synthetic polynucleotide comprising a sequence encoding (i) a CRISPR nuclease, and (ii) an epigenetic enzyme or a functional portion thereof that modifies an epigenetic state; and (b) a second synthetic polynucleotide comprising a sequence encoding a guide RNA (gRNA), wherein said engineered T-cell undergoes decreased or no T-cell exhaustion, thereby reducing or preventing T-cell exhaustion in said individual. First synthetic polynucleotide may further comprise a sequence encoding (iii) a flexible linker, wherein said linker operably links said sequence encoding (i) and (ii). Epigenetic enzyme may comprise a DNA demethylation enzyme or a DNA hydroxymethylation enzyme (e.g. TET protein such as TET1). Epigenetic enzyme may comprise a DNA methylation enzyme (e.g. DNA methyltransferase (DNMT)). Epigenetic enzyme may comprise a histone acetylation enzyme (e.g. histone acetyltransferase (HAT)). Epigenetic enzyme may comprise a histone deacetylation enzyme (e.g. histone deacetylase (HDAC)). Epigenetic enzyme may comprise a histone methylation enzyme (e.g. histone methyltransferase (HMT)). Epigenetic enzyme may comprise a histone demethylation enzyme (e.g. histone demethylase (HDM)). CRISPR nuclease may be Cas9. CRISPR nuclease may be a deactivated Cas9 (dCas9). First synthetic polynucleotide may further comprise a sequence for a constitutively active promoter. First synthetic polynucleotide may further comprise a sequence for an inducible promoter. gRNA may target a target sequence in said engineered T-cell. Target sequence may comprise a target enhancer sequence, a target regulatory element sequence, a promoter sequence of a target gene, a cis-regulatory sequence of a target gene, or a trans-regulatory sequence of a target gene. Target gene may be a gene that affects T-cell exhaustion. Targeting said target sequence may enhance function of engineered T-cell. T-cell may be a CAR T-cell. First synthetic polynucleotide and said second synthetic polynucleotide may be encoded on same vector. First synthetic polynucleotide and said second synthetic polynucleotide may be encoded on different vectors. Vector may be a viral vector or a non-viral vector.

Disclosed herein, are methods for reducing or preventing T-cell exhaustion in an individual in need thereof, said method comprising administering to said individual a cellular composition that comprises an engineered T-cell comprising: (a) a first synthetic polynucleotide comprising a sequence encoding (i) a CRISPR nuclease, and (ii) a DNA hydroxymethylation enzyme or a functional portion thereof that modifies DNA methylation state; and (b) a second synthetic polynucleotide comprising a sequence encoding a guide RNA (gRNA), wherein said engineered T-cell undergoes decreased or no T-cell exhaustion, thereby reducing or preventing T-cell exhaustion in said individual. First synthetic polynucleotide may further comprise a sequence encoding (iii) a flexible linker, wherein said linker operably links said sequence encoding (i) and (ii). Enzyme may be a TET protein such as TET1. CRISPR nuclease may be Cas9. CRISPR nuclease may be a deactivated Cas9 (dCas9). First synthetic polynucleotide may further comprise a sequence for a constitutively active promoter. First synthetic polynucleotide may further comprise a sequence for an inducible promoter. gRNA may target a target sequence in said engineered T-cell. Target sequence may comprise a target enhancer sequence, a target regulatory element sequence, a promoter sequence of a target gene, a cis-regulatory sequence of a target gene, or a trans-regulatory sequence of a target gene. Target gene may be a gene that affects T-cell exhaustion. Targeting said target sequence may enhance function of engineered T-cell. T-cell may be a CAR T-cell. First synthetic polynucleotide and said second synthetic polynucleotide may be encoded on same vector or on different vectors. Vector may be a viral vector or a non-viral vector.

Disclosed herein, are methods for providing cell therapy for treating or ameliorating a disease in an individual in need thereof, said method comprising administering to said individual a cellular composition that comprises an engineered cell comprising: (a) a first synthetic polynucleotide comprising a sequence encoding (i) a CRISPR nuclease, and (ii) an epigenetic enzyme or a functional portion thereof that modifies an epigenetic state; and (b) a second synthetic polynucleotide comprising a sequence encoding a guide RNA (gRNA). First synthetic polynucleotide may further comprise a sequence encoding (iii) a flexible linker, wherein said linker operably links said sequence encoding (i) and (ii). Epigenetic enzyme may comprise a DNA demethylation enzyme or a DNA hydroxymethylation enzyme (e.g. TET protein such as TET1). Epigenetic enzyme may comprise a DNA methylation enzyme (e.g. DNA methyltransferase (DNMT)). Epigenetic enzyme may comprise a histone acetylation enzyme (e.g. histone acetyltransferase (HAT)). Epigenetic enzyme may comprise a histone deacetylation enzyme (e.g. histone deacetylase (HDAC)). Epigenetic enzyme may comprise a histone methylation enzyme (e.g. histone methyltransferase (HMT)). Epigenetic enzyme may comprise a histone demethylation enzyme (e.g. histone demethylase (HDM)). CRISPR nuclease may be Cas9. CRISPR nuclease may be a deactivated Cas9 (dCas9). First synthetic polynucleotide may further comprise a sequence for a constitutively active promoter. First synthetic polynucleotide may further comprise a sequence for an inducible promoter. gRNA may target a target sequence in said engineered cell. Target sequence may comprise a target enhancer sequence, a target regulatory element sequence, a promoter sequence of a target gene, a cis-regulatory sequence of a target gene, or a trans-regulatory sequence of a target gene. Targeting said target sequence may enhance function of engineered cell. Cell may be a T-cell or a CAR T-Cell. Target gene may be a gene that affects T-cell exhaustion. Cellular composition may undergo decreased or no T-cell exhaustion, thereby treating or ameliorating disease in said individual. Cell may be a natural killer (NK) cell or a macrophage. First synthetic polynucleotide and said second synthetic polynucleotide may be encoded on same vector or on different vectors. Vector may be a viral vector or a non-viral vector. Disease may be cancer.

Disclosed herein, are methods for providing cell therapy for treating or ameliorating a disease in an individual in need thereof, said method comprising administering to said individual a cellular composition that comprises an engineered cell comprising: (a) a first synthetic polynucleotide comprising a sequence encoding (i) a CRISPR nuclease, and (ii) a DNA hydroxymethylation enzyme or a functional portion thereof that modifies DNA methylation state; and (b) a second synthetic polynucleotide comprising a sequence encoding a guide RNA (gRNA). First synthetic polynucleotide may further comprise a sequence encoding (iii) a flexible linker, wherein said linker operably links said sequence encoding (i) and (ii). Enzyme may be a TET protein such as TET1. CRISPR nuclease may be Cas9. CRISPR nuclease may be a deactivated Cas9 (dCas9). First synthetic polynucleotide may further comprise a sequence for a constitutively active promoter. First synthetic polynucleotide may further comprise a sequence for an inducible promoter. gRNA may target a target sequence in said engineered cell. Target sequence may comprise a target enhancer sequence, a target regulatory element sequence, a promoter sequence of a target gene, a cis-regulatory sequence of a target gene, or a trans-regulatory sequence of a target gene. Targeting said target sequence may enhance function of engineered cell. Cell may be a T-cell or a CAR T-Cell. Target gene may be a gene that affects T-cell exhaustion. Cellular composition may undergo decreased or no T-cell exhaustion, thereby treating or ameliorating disease in said individual. Cell may be a natural killer (NK) cell or a macrophage. First synthetic polynucleotide and said second synthetic polynucleotide may be encoded on same vector or on different vectors. Vector may be a viral vector or a non-viral vector. Disease may be cancer.

### DETAILED DESCRIPTION OF THE INVENTION

The methods disclosed herein provide novel tools for targeted epigenetic editing to enable maintenance of phenotypes of cell therapies. The epigenetic editing fusion proteins can be repurposed to perform targeted epigenetic maintenance in the engineered cells, with a goal of maintaining phenotypic aspects necessary for the cells to perform their desired function.

An exemplary field of use of the methods disclosed herein is T-cell exhaustion. In some instances, the T cells may be engineered to express a fusion protein like dCas9 linked to TET1, and optimized for efficacy and immunogenicity concerns. This fusion protein would be targeted to a specific genomic region that becomes methylated in the tumor microenvironment, causing an exhaustion phenotype.

In the CAR-T cells with the targeted fusion proteins, the constructs would have minimal effect as long as the target gene remains unmethylated. Whenever the target gene becomes methylated, the localized TET1 domain would reverse the effects, ensure continued protein expression, and prevent the engineered T cells from becoming exhausted. The dCas9-TET1 ensures that, for the target gene, the methylation state that the cells are administered with will be maintained.

This cell therapy technology can be applied across multiple cell types and target genes, and can be constructed using multiple gene targeting domains, linkers, epigenetic modifiers, and transfection and expression strategies.

Cell types could include T cells (including CAR-T cells, or T cells with engineered TCRs (alpha beta or gamma delta)), Natural Killer cells, Natural Killer T cells, macrophages, or other cells that are engineered and subsequently administered as a treatment.

The flexibility of the targeting moieties of the fusion proteins allows for a range of different potential targets, from specific genes or promotor regions to regions selected for their ability to alter gene loops or other higher-level chromatin structural changes. Taking the specific gene example, the expression level could be maintained in an on or off state depending on the selected epigenetic modifier.

The targeting component of the fusion protein could be built from modified CRISPR-associated proteins, transcription activator-like effectors (TALEs), zinc fingers, transcription factors, or other constructs with the ability to target specific areas of the genome.

The linker component could be a flexible peptide linker, a rigid peptide linker, a cleavable peptide linker, or a non-peptide molecule used to connect the targeting and effector domains of the construct.

The epigenetic modifiers could include proteins that alter DNA methylation states (e.g. DNMTs, TETs, etc.), proteins that modify histones (e.g. histone deacetylases, histone acetyltransferases, etc.), or versions engineered to decrease size or increase efficacy.

The engineered cells could either constitutively express these fusion proteins or be transiently transfected. For constitutive expression, a number of different delivery approaches for the genetic material could be used depending on the cell type, ranging from viral transfection to electroporation of RNPs.

In some instances, the methods for genetic modification comprise any of the gene editing tools disclosed herein.

### Targetable Nucleic Acid Cleavage Systems

Methods disclosed herein comprise targeting cleavage of specific nucleic acid sequences using a site-specific, targetable, and/or engineered nuclease or nuclease system. Such nucleases may create double-stranded break (DSBs) at desired locations in a genome or nucleic acid molecule. In other examples, a nuclease may create a single strand break. In some cases, two nucleases are used, each of which generates a single strand break.

The one or more double or single strand break may be repaired by natural processes of homologous recombination (HR) and non-homologous end-joining (NHEJ) using the cell's endogenous machinery. Additionally or alternatively, endogenous or heterologous recombination machinery may be used to repair the induced break or breaks.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems may be used, such as CRISPR/Cas systems including c2c2 nucleases.

Methods disclosed herein may comprise cleaving a target nucleic acid using CRISPR systems, such as a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. Alternatively, Class 2 systems include a single effector molecule and include Type II, Type V, and Type VI.

CRISPR systems used in methods disclosed herein may comprise a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

A gRNA may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence may be less than 10 or more than 36 nucleotides in length.

A gRNA may comprise a repeat sequence. In some cases, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence may be less than 10 or more than 50 nucleotides in length.

A gRNA may comprise one or more synthetic nucleotides, non-naturally occurring nucleotides, nucleotides with a modification, deoxyribonucleotide, or any combination thereof. Additionally or alternatively, a gRNA may comprise a hairpin, linker region, single stranded region, double stranded region, or any combination thereof. Additionally or alternatively, a gRNA may comprise a signaling or reporter molecule.

A CRISPR nuclease may be endogenously or recombinantly expressed within a cell. A CRISPR nuclease may be encoded on a chromosome, extrachromosomally, or on a plasmid, synthetic chromosome, or artificial chromosome. A CRISPR nuclease may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

gRNAs may be encoded by genetic or episomal DNA within a cell. In some examples, gRNAs may be provided or delivered to a cell expressing a CRISPR nuclease. gRNAs may be provided or delivered concomitantly with a CRISPR nuclease or sequentially. Guide RNAs may be chemically synthesized, in vitro transcribed or otherwise generated using standard RNA generation techniques known in the art.

A CRISPR system may be a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM may be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which is some cases creates two blunt ends. In some cases, the Type II CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease may be catalytically dead such that it binds to a target sequence, but does not cleave. For example, a Type II nuclease may have mutations in both the RuvC and HNH domains, thereby rendering the both nuclease domains non-functional. A Type II CRISPR system may be one of three sub-types, namely Type II-A, Type II-B, or Type II-C.

A CRISPR system may be a Type V CRISPR system, for example a Cpf1, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease may be catalytically dead such that it binds to a target sequence, but does not cleave. For example, a Type V nuclease could have mutations a RuvC domain, thereby rendering the nuclease domain non-functional.

A CRISPR system may be a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems may be used to cleave target nucleic acid sequences. Ago protein may be derived from a prokaryote, eukaryote, or archaea. The target nucleic acid may be RNA or DNA. A DNA target may be single stranded or double stranded. In some examples, the target nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins may be used in combination to generate a double strand break. In some examples, an Ago protein comprises one, two, or more nuclease domains. In some examples, an Ago protein comprises one, two, or more catalytic domains. One or more nuclease or catalytic domains may be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In many examples, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA may be less than 10 nucleotides in length. In some examples, the gDNA may be more than 50 nucleotides in length.

Argonaute-mediated cleavage may generate blunt end, 5′ overhangs, or 3′ overhangs. In some examples, one or more nucleotides are removed from the target site during or following cleavage.

Argonaute protein may be endogenously or recombinantly expressed within a cell. Argonaute may be encoded on a chromosome, extrachromosomally, or on a plasmid, synthetic chromosome, or artificial chromosome. Additionally or alternatively, an Argonaute protein may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

Guide DNAs may be provided by genetic or episomal DNA within a cell. In some examples, gDNA are reverse transcribed from RNA or mRNA within a cell. In some examples, gDNAs may be provided or delivered to a cell expressing an Ago protein. Guide DNAs may be provided or delivered concomitantly with an Ago protein or sequentially. Guide DNAs may be chemically synthesized, assembled, or otherwise generated using standard DNA generation techniques known in the art. Guide DNAs may be cleaved, released, or otherwise derived from genomic DNA, episomal DNA molecules, isolated nucleic acid molecules, or any other source of nucleic acid molecules.

Nuclease fusion proteins may be recombinantly expressed within a cell. A nuclease fusion protein may be encoded on a chromosome, extrachromosomally, or on a plasmid, synthetic chromosome, or artificial chromosome. A nuclease and a chromatin-remodeling enzyme may be engineered separately, and then covalently linked, prior to delivery to a cell. A nuclease fusion protein may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

### Guide Nucleic Acid

A guide nucleic acid may complex with a compatible nucleic acid-guided nuclease and may hybridize with a target sequence, thereby directing the nuclease to the target sequence. A subject nucleic acid-guided nuclease capable of complexing with a guide nucleic acid may be referred to as a nucleic acid-guided nuclease that is compatible with the guide nucleic acid. Likewise, a guide nucleic acid capable of complexing with a nucleic acid-guided nuclease may be referred to as a guide nucleic acid that is compatible with the nucleic acid-guided nucleases.

A guide nucleic acid may be DNA. A guide nucleic acid may be RNA. A guide nucleic acid may comprise both DNA and RNA. A guide nucleic acid may comprise modified of non-naturally occurring nucleotides. In cases where the guide nucleic acid comprises RNA, the RNA guide nucleic acid may be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence may be 10-25 nucleotides in length. The guide sequence may be 10-20 nucleotides in length. The guide sequence may be 15-30 nucleotides in length. The guide sequence may be 20-30 nucleotides in length. The guide sequence may be 15-25 nucleotides in length. The guide sequence may be 15-20 nucleotides in length. The guide sequence may be 20-25 nucleotides in length. The guide sequence may be 22-25 nucleotides in length. The guide sequence may be 15 nucleotides in length. The guide sequence may be 16 nucleotides in length. The guide sequence may be 17 nucleotides in length. The guide sequence may be 18 nucleotides in length. The guide sequence may be 19 nucleotides in length. The guide sequence may be 20 nucleotides in length. The guide sequence may be 21 nucleotides in length. The guide sequence may be 22 nucleotides in length. The guide sequence may be 23 nucleotides in length. The guide sequence may be 24 nucleotides in length. The guide sequence may be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions may be 10-25 nucleotides in length. At least one of the two sequence regions may be 10-20 nucleotides in length. At least one of the two sequence regions may be 15-30 nucleotides in length. At least one of the two sequence regions may be 20-30 nucleotides in length. At least one of the two sequence regions may be 15-25 nucleotides in length. At least one of the two sequence regions may be 15-20 nucleotides in length. At least one of the two sequence regions may be 20-25 nucleotides in length. At least one of the two sequence regions may be 22-25 nucleotides in length. At least one of the two sequence regions may be 15 nucleotides in length. At least one of the two sequence regions may be 16 nucleotides in length. At least one of the two sequence regions may be 17 nucleotides in length. At least one of the two sequence regions may be 18 nucleotides in length. At least one of the two sequence regions may be 19 nucleotides in length. At least one of the two sequence regions may be 20 nucleotides in length. At least one of the two sequence regions may be 21 nucleotides in length. At least one of the two sequence regions may be 22 nucleotides in length. At least one of the two sequence regions may be 23 nucleotides in length. At least one of the two sequence regions may be 24 nucleotides in length. At least one of the two sequence regions may be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In aspects of the disclosure the terms “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid may be compatible with a nucleic acid-guided nuclease when the two elements may form a functional targetable nuclease complex capable of cleaving a target sequence. Often, a compatible scaffold sequence for a compatible guide nucleic acid may be found by scanning sequences adjacent to native nucleic acid-guided nuclease loci. In other words, native nucleic acid-guided nucleases may be encoded on a genome within proximity to a corresponding compatible guide nucleic acid or scaffold sequence.

Nucleic acid-guided nucleases may be compatible with guide nucleic acids that are not found within the nucleases endogenous host. Such orthogonal guide nucleic acids may be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid may be engineered to target a desired target sequence by altering the guide sequence such that the guide sequence is complementary to the target sequence, thereby allowing hybridization between the guide sequence and the target sequence. A guide nucleic acid with an engineered guide sequence may be referred to as an engineered guide nucleic acid. Engineered guide nucleic acids are often non-naturally occurring and are not found in nature.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for providing cell therapy for treating or ameliorating a disease in an individual in need thereof, said method comprising administering to said individual a cellular composition that comprises an engineered T-cell comprising:
(a) a first synthetic polynucleotide comprising a sequence encoding (i) a CRISPR nuclease, and (ii) an epigenetic enzyme or a functional portion thereof that modifies an epigenetic state; and
(b) a second synthetic polynucleotide comprising a sequence encoding a guide RNA (gRNA).

2. The method of claim 1, wherein said first synthetic polynucleotide further comprises a sequence encoding (iii) a flexible linker, wherein said linker operably links said sequence encoding (i) and (ii).

3. The method of claim 1, wherein said epigenetic enzyme comprises a DNA demethylation enzyme.

4. The method of claim 1, wherein said epigenetic enzyme comprises a DNA hydroxymethylation enzyme.

5. The method of claim 3 or claim 4, wherein said enzyme is a TET protein.

6. The method of claim 5, wherein said TET protein is TET1.

7. The method of claim 1, wherein said epigenetic enzyme comprises a DNA methylation enzyme.

8. The method of claim 7, wherein said DNA methylation enzyme is DNA methyltransferase (DNMT).

9. The method of claim 1, wherein said epigenetic enzyme comprises a histone acetylation enzyme.

10. The method of claim 9, wherein said histone acetylation enzyme is histone acetyltransferase (HAT).

11. The method of claim 1, wherein said epigenetic enzyme comprises a histone deacetylation enzyme.

12. The method of claim 11, wherein said histone deacetylation enzyme is histone deacetylase (HDAC).

13. The method of claim 1, wherein said epigenetic enzyme comprises a histone methylation enzyme.

14. The method of claim 13, wherein said histone methylation enzyme is histone methyltransferase (HMT).

15. The method of claim 1, wherein said epigenetic enzyme comprises a histone demethylation enzyme.

16. The method of claim 15, wherein said histone demethylation enzyme is histone demethylase (HDM).

17. The method of claim 1, wherein said CRISPR nuclease is Cas9.

18. The method of claim 1, wherein said CRISPR nuclease is a deactivated Cas9 (dCas9).

19. The method of claim 1, wherein said first synthetic polynucleotide further comprises a sequence for a constitutively active promoter.

20. The method of claim 1, wherein said first synthetic polynucleotide further comprises a sequence for an inducible promoter.

21. The method of claim 1, wherein said gRNA targets a target sequence in said engineered T-cell.

22. The method of claim 21, wherein said target sequence comprises a target enhancer sequence, a target regulatory element sequence, a promoter sequence of a target gene, a cis-regulatory sequence of a target gene, or a trans-regulatory sequence of a target gene.

23. The method of claim 22, wherein said target gene is a gene that affects T-cell exhaustion.

24. The method of claim 21, wherein targeting said target sequence enhances function of engineered T-cell.

25. The method of claim 1, wherein administering said cellular composition undergoes decreased or no T-cell exhaustion, thereby treating or ameliorating disease in said individual.

26. The method of claim 1, wherein said T-cell is a CAR T-cell.

27. The method of claim 1, wherein said first synthetic polynucleotide and said second synthetic polynucleotide are encoded on same vector.

28. The method of claim 1, wherein said first synthetic polynucleotide and said second synthetic polynucleotide are encoded on different vectors.

29. The method of claim 1, wherein said vector is a viral vector.

30. The method of claim 1, wherein said vector is a non-viral vector.

31. The method of claim 1, wherein said disease is cancer.

32. A method for providing cell therapy for treating or ameliorating a disease in an individual in need thereof, said method comprising administering to said individual a cellular composition that comprises an engineered T-cell comprising:
(a) a first synthetic polynucleotide comprising a sequence encoding (i) a CRISPR nuclease, and (ii) a DNA hydroxymethylation enzyme or a functional portion thereof that modifies DNA methylation state; and
(b) a second synthetic polynucleotide comprising a sequence encoding a guide RNA (gRNA).

33. The method of claim 32, wherein said first synthetic polynucleotide further comprises a sequence encoding (iii) a flexible linker, wherein said linker operably links said sequence encoding (i) and (ii).

34. The method of claim 32, wherein said enzyme is a TET protein.

35. The method of claim 34, wherein said TET protein is TET1.

36. The method of claim 32, wherein said CRISPR nuclease is Cas9.

37. The method of claim 32, wherein said CRISPR nuclease is a deactivated Cas9 (dCas9).

38. The method of claim 32, wherein said first synthetic polynucleotide further comprises a sequence for a constitutively active promoter.

39. The method of claim 32, wherein said first synthetic polynucleotide further comprises a sequence for an inducible promoter.

40. The method of claim 32, wherein said gRNA targets a target sequence in said engineered T-cell.

41. The method of claim 40, wherein said target sequence comprises a target enhancer sequence, a target regulatory element sequence, a promoter sequence of a target gene, a cis-regulatory sequence of a target gene, or a trans-regulatory sequence of a target gene.

42. The method of claim 41, wherein said target gene is a gene that affects T-cell exhaustion.

43. The method of claim 40, wherein targeting said target sequence enhances function of engineered T-cell.

44. The method of claim 32, wherein administering said cellular composition undergoes decreased or no T-cell exhaustion, thereby treating or ameliorating disease in said individual.

45. The method of claim 32, wherein said T-cell is a CAR T-cell.

46. The method of claim 32, wherein said first synthetic polynucleotide and said second synthetic polynucleotide are encoded on same vector.

47. The method of claim 32, wherein said first synthetic polynucleotide and said second synthetic polynucleotide are encoded on different vectors.

48. The method of claim 32, wherein said vector is a viral vector.

49. The method of claim 32, wherein said vector is a non-viral vector.

50. The method of claim 32, wherein said disease is cancer.

51. A method for reducing or preventing T-cell exhaustion in an individual in need thereof, said method comprising administering to said individual a cellular composition that comprises an engineered T-cell comprising:
(a) a first synthetic polynucleotide comprising a sequence encoding (i) a CRISPR nuclease, and (ii) an epigenetic enzyme or a functional portion thereof that modifies an epigenetic state; and
(b) a second synthetic polynucleotide comprising a sequence encoding a guide RNA (gRNA),
wherein said engineered T-cell undergoes decreased or no T-cell exhaustion, thereby reducing or preventing T-cell exhaustion in said individual.

52. The method of claim 51, wherein said first synthetic polynucleotide further comprises a sequence encoding (iii) a flexible linker, wherein said linker operably links said sequence encoding (i) and (ii).

53. The method of claim 51, wherein said epigenetic enzyme comprises a DNA demethylation enzyme.

54. The method of claim 51, wherein said epigenetic enzyme comprises a DNA hydroxymethylation enzyme.

55. The method of claim 53 or claim 54, wherein said enzyme is a TET protein.

56. The method of claim 55, wherein said TET protein is TET1.

57. The method of claim 51, wherein said epigenetic enzyme comprises a DNA methylation enzyme.

58. The method of claim 57, wherein said DNA methylation enzyme is DNA methyltransferase (DNMT).

59. The method of claim 51, wherein said epigenetic enzyme comprises a histone acetylation enzyme.

60. The method of claim 59, wherein said histone acetylation enzyme is histone acetyltransferase (HAT).

61. The method of claim 51, wherein said epigenetic enzyme comprises a histone deacetylation enzyme.

62. The method of claim 61, wherein said histone deacetylation enzyme is histone deacetylase (HDAC).

63. The method of claim 51, wherein said epigenetic enzyme comprises a histone methylation enzyme.

64. The method of claim 63, wherein said histone methylation enzyme is histone methyltransferase (HMT).

65. The method of claim 51, wherein said epigenetic enzyme comprises a histone demethylation enzyme.

66. The method of claim 65, wherein said histone demethylation enzyme is histone demethylase (HDM).

67. The method of claim 51, wherein said CRISPR nuclease is Cas9.

68. The method of claim 51, wherein said CRISPR nuclease is a deactivated Cas9 (dCas9).

69. The method of claim 51, wherein said first synthetic polynucleotide further comprises a sequence for a constitutively active promoter.

70. The method of claim 51, wherein said first synthetic polynucleotide further comprises a sequence for an inducible promoter.

71. The method of claim 51, wherein said gRNA targets a target sequence in said engineered T-cell.

72. The method of claim 71, wherein said target sequence comprises a target enhancer sequence, a target regulatory element sequence, a promoter sequence of a target gene, a cis-regulatory sequence of a target gene, or a trans-regulatory sequence of a target gene.

73. The method of claim 72, wherein said target gene is a gene that affects T-cell exhaustion.

74. The method of claim 71, wherein targeting said target sequence enhances function of engineered T-cell.

75. The method of claim 51, wherein said T-cell is a CAR T-cell.

76. The method of claim 51, wherein said first synthetic polynucleotide and said second synthetic polynucleotide are encoded on same vector.

77. The method of claim 51, wherein said first synthetic polynucleotide and said second synthetic polynucleotide are encoded on different vectors.

78. The method of claim 51, wherein said vector is a viral vector.

79. The method of claim 51, wherein said vector is a non-viral vector.

80. A method for reducing or preventing T-cell exhaustion in an individual in need thereof, said method comprising administering to said individual a cellular composition that comprises an engineered T-cell comprising:
(a) a first synthetic polynucleotide comprising a sequence encoding (i) a CRISPR nuclease, and (ii) a DNA hydroxymethylation enzyme or a functional portion thereof that modifies DNA methylation state; and
(b) a second synthetic polynucleotide comprising a sequence encoding a guide RNA (gRNA),
wherein said engineered T-cell undergoes decreased or no T-cell exhaustion, thereby reducing or preventing T-cell exhaustion in said individual.

81. The method of claim 80, wherein said first synthetic polynucleotide further comprises a sequence encoding (iii) a flexible linker, wherein said linker operably links said sequence encoding (i) and (ii).

82. The method of claim 80, wherein said enzyme is a TET protein.

83. The method of claim 82, wherein said TET protein is TET1.

84. The method of claim 80, wherein said CRISPR nuclease is Cas9.

85. The method of claim 80, wherein said CRISPR nuclease is a deactivated Cas9 (dCas9).

86. The method of claim 80, wherein said first synthetic polynucleotide further comprises a sequence for a constitutively active promoter.

87. The method of claim 80, wherein said first synthetic polynucleotide further comprises a sequence for an inducible promoter.

88. The method of claim 80, wherein said gRNA targets a target sequence in said engineered T-cell.

89. The method of claim 88, wherein said target sequence comprises a target enhancer sequence, a target regulatory element sequence, a promoter sequence of a target gene, a cis-regulatory sequence of a target gene, or a trans-regulatory sequence of a target gene.

90. The method of claim 89, wherein said target gene is a gene that affects T-cell exhaustion.

91. The method of claim 88, wherein targeting said target sequence enhances function of engineered T-cell.

92. The method of claim 80, wherein said T-cell is a CAR T-cell.

93. The method of claim 80, wherein said first synthetic polynucleotide and said second synthetic polynucleotide are encoded on same vector.

94. The method of claim 80, wherein said first synthetic polynucleotide and said second synthetic polynucleotide are encoded on different vectors.

95. The method of claim 80, wherein said vector is a viral vector.

96. The method of claim 80, wherein said vector is a non-viral vector.

97. A method for providing cell therapy for treating or ameliorating a disease in an individual in need thereof, said method comprising administering to said individual a cellular composition that comprises an engineered cell comprising:
(a) a first synthetic polynucleotide comprising a sequence encoding (i) a CRISPR nuclease, and (ii) an epigenetic enzyme or a functional portion thereof that modifies an epigenetic state; and
(b) a second synthetic polynucleotide comprising a sequence encoding a guide RNA (gRNA).

98. The method of claim 97, wherein said first synthetic polynucleotide further comprises a sequence encoding (iii) a flexible linker, wherein said linker operably links said sequence encoding (i) and (ii).

99. The method of claim 97, wherein said epigenetic enzyme comprises a DNA demethylation enzyme.

100. The method of claim 97, wherein said epigenetic enzyme comprises a DNA hydroxymethylation enzyme.

101. The method of claim 99 or claim 100, wherein said enzyme is a TET protein.

102. The method of claim 101, wherein said TET protein is TET1.

103. The method of claim 97, wherein said epigenetic enzyme comprises a DNA methylation enzyme.

104. The method of claim 103, wherein said DNA methylation enzyme is DNA methyltransferase (DNMT).

105. The method of claim 97, wherein said epigenetic enzyme comprises a histone acetylation enzyme.

106. The method of claim 105, wherein said histone acetylation enzyme is histone acetyltransferase (HAT).

107. The method of claim 97, wherein said epigenetic enzyme comprises a histone deacetylation enzyme.

108. The method of claim 107, wherein said histone deacetylation enzyme is histone deacetylase (HDAC).

109. The method of claim 97, wherein said epigenetic enzyme comprises a histone methylation enzyme.

110. The method of claim 109, wherein said histone methylation enzyme is histone methyltransferase (HMT).

111. The method of claim 97, wherein said epigenetic enzyme comprises a histone demethylation enzyme.

112. The method of claim 111, wherein said histone demethylation enzyme is histone demethylase (HDM).

113. The method of claim 97, wherein said CRISPR nuclease is Cas9.

114. The method of claim 97, wherein said CRISPR nuclease is a deactivated Cas9 (dCas9).

115. The method of claim 97, wherein said first synthetic polynucleotide further comprises a sequence for a constitutively active promoter.

116. The method of claim 97, wherein said first synthetic polynucleotide further comprises a sequence for an inducible promoter.

117. The method of claim 97, wherein said gRNA targets a target sequence in said engineered cell.

118. The method of claim 117, wherein said target sequence comprises a target enhancer sequence, a target regulatory element sequence, a promoter sequence of a target gene, a cis-regulatory sequence of a target gene, or a trans-regulatory sequence of a target gene.

119. The method of claim 117, wherein targeting said target sequence enhances function of engineered cell.

120. The method of claim 97, wherein said cell is a T-cell.

121. The method of claim 120, wherein said T-cell is a CAR T-cell.

122. The method of claim 120, wherein said target gene is a gene that affects T-cell exhaustion.

123. The method of claim 120, wherein administering said cellular composition undergoes decreased or no T-cell exhaustion, thereby treating or ameliorating disease in said individual.

124. The method of claim 97, wherein said cell is a natural killer (NK) cell.

125. The method of claim 97, wherein said cell is a macrophage.

126. The method of claim 97, wherein said first synthetic polynucleotide and said second synthetic polynucleotide are encoded on same vector.

127. The method of claim 97, wherein said first synthetic polynucleotide and said second synthetic polynucleotide are encoded on different vectors.

128. The method of claim 97, wherein said vector is a viral vector.

129. The method of claim 97, wherein said vector is a non-viral vector.

130. The method of claim 97, wherein said disease is cancer.

131. A method for providing cell therapy for treating or ameliorating a disease in an individual in need thereof, said method comprising administering to said individual a cellular composition that comprises an engineered cell comprising:
(a) a first synthetic polynucleotide comprising a sequence encoding (i) a CRISPR nuclease, and (ii) a DNA hydroxymethylation enzyme or a functional portion thereof that modifies DNA methylation state; and
(b) a second synthetic polynucleotide comprising a sequence encoding a guide RNA (gRNA).

132. The method of claim 131, wherein said first synthetic polynucleotide further comprises a sequence encoding (iii) a flexible linker, wherein said linker operably links said sequence encoding (i) and (ii).

133. The method of claim 131, wherein said enzyme is a TET protein.

134. The method of claim 133, wherein said TET protein is TET1.

135. The method of claim 131, wherein said CRISPR nuclease is Cas9.

136. The method of claim 131, wherein said CRISPR nuclease is a deactivated Cas9 (dCas9).

137. The method of claim 131, wherein said first synthetic polynucleotide further comprises a sequence for a constitutively active promoter.

138. The method of claim 131, wherein said first synthetic polynucleotide further comprises a sequence for an inducible promoter.

139. The method of claim 131, wherein said gRNA targets a target sequence in said engineered cell.

140. The method of claim 139, wherein said target sequence comprises a target enhancer sequence, a target regulatory element sequence, a promoter sequence of a target gene, a cis-regulatory sequence of a target gene, or a trans-regulatory sequence of a target gene.

141. The method of claim 139, wherein targeting said target sequence enhances function of engineered cell.

142. The method of claim 131, wherein said cell is a T-cell.

143. The method of claim 142, wherein said T-cell is a CAR T-cell.

144. The method of claim 142, wherein said target gene is a gene that affects T-cell exhaustion.

145. The method of claim 142, wherein administering said cellular composition undergoes decreased or no T-cell exhaustion, thereby treating or ameliorating disease in said individual.

146. The method of claim 131, wherein said cell is a natural killer (NK) cell.

147. The method of claim 131, wherein said cell is a macrophage.

148. The method of claim 131, wherein said first synthetic polynucleotide and said second synthetic polynucleotide are encoded on same vector.

149. The method of claim 131, wherein said first synthetic polynucleotide and said second synthetic polynucleotide are encoded on different vectors.

150. The method of claim 131, wherein said vector is a viral vector.

151. The method of claim 131, wherein said vector is a non-viral vector.

152. The method of claim 131, wherein said disease is cancer.
